(19) United States
(12) Patent Application Publication
(10) Pub. No.: US 2025/0255760 A1
(43) Pub. Date: Aug. 14, 2025

(54) Wearable Garment for Reducing Bacterial Growth

(71) Applicant: Jacqueline Krieger, San Clemente, CA (US)

(72) Inventor: Jacqueline Krieger, San Clemente, CA (US)

(21) Appl. No.: 18/440,120

(22) Filed: Feb. 13, 2024

(51) Int. Cl. A61F 13/84; A61F 13/495; A61F 13/496; A61F 13/511

(52) U.S. Cl. CPC A61F 13/8405; A61F 13/495; A61F 13/496; A61F 13/5116; A61F 2013/4958; A61F 2013/8476

(57) ABSTRACT

An undergarment comprising a liquid impermeable layer which prevents urine, fluid, and any other media capable of transporting and breeding bacteria that is deposited into the undergarment from coming into contact with the skin of the user or person wearing the undergarment. The fluid that is released from the wearer makes contact with a topsheet and is then transferred to a top facing surface of the liquid impermeable layer. The curvature or shape of the liquid impermeable layer directs the fluid away from the skin of the wearer and towards its outer edges. Once reaching an edge of the liquid impermeable layer, the fluid is then absorbed and held within an absorbent core. By maintaining the fluid within the absorbent core, the liquid impermeable layer prevents the fluid and any bacteria therein from making sustained contact with the wearer's anatomy and thus drastically lowers the probability of infection or other complications.

### BACKGROUND

### Field of the Technology

The invention relates to the field of wearable undergarments. These undergarments are worn by adults, children, or infants and may reduce the growth of bacteria and infections arising therefrom.

### Description of the Prior Art

Wearable undergarments such as diapers, sanitary napkins, incontinent pads, or the like have long been used to collect or hold waste for individuals who are either too young or otherwise incapable of relieving themselves in a restroom or other appropriate facility. Many of these undergarments comprise multiple layers, including an absorbent core sandwiched or disposed between a topsheet and a backsheet. Many undergarments also comprise a distribution layer that draws liquid and other waste away from the topsheet so that it may more quickly make contact with the absorbent core.

A number of specialty undergarments that are principally worn by medical patients have also been developed. These include an opening or other means for accommodating a stool sample that may be accessed later for testing purposes. Additionally, some undergarments comprise a stool blocker, which limits the movement of the stool within the undergarment and reduces contaminating an area of the undergarment used for urine collection or sampling.

However, many common undergarments are configured such that once that waste is collected within them, the undergarment will maintain and hold the waste next to the wearer's skin. Furthermore, undergarment layers between the wearer's skin and the absorbent core can allow for the backflow of moisture, waste, and bacteria to reach the wearer's skin. If the undergarment is not frequently changed or if the wearer is not otherwise routinely cleaned, waste such as urine or fecal matter can promote the breeding of bacteria on the user's skin, leading to infection, rashes, or other types of irritation. This can be problematic when the undergarment is worn for extended periods of time, such as in the elderly who are dependent on others to maintain their sanitary needs or for people who are otherwise physically incapacitated.

Therefore, an undergarment which reduces the growth of bacteria which, if left untreated or unaddressed, can later cause irritation or infection, is needed. The undergarment should be relatively comfortable and be easy enough to use so that no additional training is required.

### BRIEF SUMMARY

The current invention provides an undergarment or diaper which helps reduce the incidence of urinary tract infections in the wearer. According to some embodiments, the current invention reduces the concentration and growth of bacteria held under the urethral opening of the wearer. Compared to men, women are at an increased risk of urinary tract infection (UTI) due to their anatomically shorter urethras. A shorter urethra makes it easier for bacteria to ascend into the urinary system and cause infection. In women who wear diapers/incontinence products, this risk of UTI is further increased by the diaper holding bacteria containing waste close to the body for prolonged periods of time. Men are also at risk for such infections, though they tend to suffer infection less frequently than women.

In some embodiments, the current undergarment comprises a substantially liquid impermeable or hydrophobic UTI guard. According to some embodiments, the guard layer is completely impermeable, however in additional embodiments the layer is largely or more than 50% impermeable or has different degrees of permeability and hydrophobicity. For ease of descriptive purposes, this layer will be referred to as a liquid impermeable or hydrophobic layer, but it is to be understood that the layer can have a range of varied impermeability and hydrophobicity. In another embodiment, the guard layer is gas-permeable to allow breathability. According to certain other embodiments, the undergarment comprises a “stool guard” which is configured to keep stool away from anatomical features of the wearer's body that are prone to infection or irritation.

The current invention provides a removable undergarment for directing fluid away from a wearer. According to some embodiments, the apparatus comprises a topsheet, an absorbent core, and a liquid impermeable layer disposed beneath the topsheet and above the absorbent core, wherein the liquid impermeable layer comprises a smaller surface area relative to the topsheet.

In one embodiment, the liquid impermeable layer comprises a plurality of grooves configured to direct fluid to at least one outer edge of the liquid impermeable layer.

In another embodiment, the liquid impermeable layer is comprised of a liquid impermeable material, such as polyethylene, polyester, polypropylene, polyurethane, polyvinyl chloride, ethylene-vinyl acetate, polytetrafluoroethylene, expanded polytetrafluoroethylene, Gore-Tex®, or silicone. In a related embodiment, the liquid impermeable layer is also comprised of any known polymer, elastomer, rubber, composite, fabric, woven fiber, spunbound fiber, membrane, film, coating or layer that imparts a degree of liquid impermeability or hydrophobicity, or both. According to certain embodiments, the layer may also comprise other now known or later devised materials suitable for such purposes listed above.

In yet another embodiment, the liquid impermeable layer comprises at least one of the following materials or coatings: antibacterial material, antiviral material, bacteriostatic material, bactericidal material, antifungal material, moisturizing material, or infection indicating material. According to certain embodiments, the liquid impermeable layer comprises materials including silver, copper, zinc oxide, molybdenum disulfide, nitride, pyrithione sodium, antibiotics, antimicrobial peptides, polymers, or essential oils. It is to be expressly understood that the materials or coatings disposed on the liquid impermeable layer and the layer itself can also be comprised of additional now known or later devised materials or coatings, as well as proprietary compounds or surface technologies devised for imparting antimicrobial properties, biofilm destruction, or microbial sterilization.

In yet another embodiment, the liquid impermeable layer comprises visible light phototherapy to act as an antibacterial and antimicrobial agent. The visible light phototherapy is delivered using a thin strip of light emitting diode (LED) lights. The strip is flexible and waterproof and is disposed on, embedded into, or otherwise coupled to the bottom surface of the impermeable layer. The LED provides light in various wavelengths that kill microorganisms. The light intermittently or constantly illuminates the impermeable layer, topsheet, or the absorbent core to reduce microbial growth. According to one embodiment, the light device is a disposable or reusable LED light with a small battery component. The device is disposed on the bottom of the liquid impermeable layer and is oriented or directed downward towards the absorbent core. In an alternative embodiment, the impermeable layer comprises anti-microbial lights facing both upwards towards the topsheet and simultaneously downwards towards to absorbent core below to provide light in both directions. The strips are oriented so as to either entirely cover or partially cover the bottom of the liquid impermeable membrane.

In one embodiment, the liquid impermeable layer comprises a device that uses a plurality of tubes and connectors to deliver antimicrobial substances or drying materials to the liquid impermeable layer to reduce bacterial growth and promote dryness. A different plurality of tubes and connectors then remove the discarded substances. In some embodiments, the antimicrobial substance delivered to the liquid impermeable layer comprises antimicrobial cleansers, disinfectant, ozone, or air to keep the impermeable layer dry. According to one embodiment, a leur connector, or other connector, on the exterior of the undergarment is coupled to the impermeable layer and allows the passage of liquid or gas material to the impermeable layer with the purpose of drying the layer or sanitizing. Once used, the instilled fluid or gas exits into an exterior bag or through a second luer connector and away from the wearer. Furthermore, this exit port can be used with an external pH sensor or bacterial sensor to quickly assess for bacteria or infection.

According to one embodiment, the liquid impermeable layer is disposed within a front portion of the undergarment. Here, the undergarment may also comprise a stool blocker that is disposed between the front and back portions of the undergarment.

In one embodiment, the liquid impermeable layer comprises a shape that is configured to be disposed directly beneath the vaginal and urethral openings if the person wearing the undergarment is female, and directly beneath the glans of the penis and urethra if the person wearing the undergarment is male.

In an additional embodiment, the undergarment also comprises a border area that is adjacent to the liquid impermeable layer. The border area provides direct fluid communication between the topsheet and the absorbent core so waste that hits the liquid impermeable layer can roll off the sides and does not pool on the liquid impermeable layer.

In an alternative embodiment, the liquid impermeable layer comprises at least one aperture defined therein, with at least one aperture being specifically configured to direct fluid through the liquid impermeable layer.

In another embodiment, the undergarment also comprises a distribution layer that is disposed between the liquid impermeable layer and the absorbent core.

In yet another embodiment, the undergarment further includes a distribution layer that is disposed between the topsheet and the liquid impermeable layer or in-line with the liquid impermeable layer.

The invention further provides a method for reducing bacterial infections in an individual wearing a removable undergarment. According to certain embodiments, the method comprises contacting a topsheet of the undergarment to a portion of the wearer's anatomy, absorbing a fluid received from the wearer through the topsheet, and then directing the received fluid across a surface of a liquid impermeable layer and to at least one outer edge of the liquid impermeable layer. The fluid is then absorbed within a core disposed beneath the liquid impermeable layer.

In one embodiment, the step of directing the received fluid across the surface of the liquid impermeable layer comprises directing the received fluid through at least one channel defined on the surface of the liquid impermeable layer.

In one embodiment, the step of directing the received fluid across the surface of the liquid impermeable layer comprises directing the received fluid across a front portion of the undergarment.

In another embodiment, the step of directing the received fluid to at least one outer edge of the liquid impermeable layer comprises directing the received fluid to a border area that is adjacent to the liquid impermeable layer and beneath the topsheet.

In a further embodiment, directing the received fluid across the surface of the liquid impermeable layer comprises directing the received fluid through at least one aperture that is defined through the liquid impermeable layer.

In another embodiment, the method also includes blocking stool received from the wearer from entering the front portion of the undergarment.

In a related embodiment, the method further includes preventing fluid that has been absorbed by the core from recontacting the topsheet that is in contact with the portion of the wearer's anatomy.

In another embodiment, the method also comprises distributing the fluid over a surface of the core after it has been directed off of at least one outer edge of the liquid impermeable layer.

According to one embodiment, the method also includes distributing the received fluid over the surface of the liquid impermeable layer after it has been absorbed through the topsheet.

While the apparatus and method have or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a perspective partially cross-sectional view of a diaper of

### THE PRIOR ART

FIG. 2 is a top-down plan view of the undergarment of the current invention wherein the relative location of a liquid impermeable layer is shown in broken line outline.

FIG. 3 is a magnified side cross-sectional view of the undergarment of FIG. 2.

FIG. 4 is a magnified side perspective view of an alternative embodiment of the undergarment of FIG. 2, wherein the undergarment further comprises a stool blocker.

FIG. 5 is a top-down plan view of the undergarment of FIG. 4.

FIG. 6A is a frontal perspective view of various shapes of the liquid impermeable layers of the current invention. Shown is the liquid impermeable layer having a substantially rectangular shape, a substantially square shape, and a substantially triangular shape.

FIG. 6B is a top-down plan view of an alternative liquid impermeable layer of the current invention, the liquid impermeable layer having a substantially oval shape.

FIG. 7 is a magnified top-down view of one embodiment of the liquid impermeable layer of the current invention, which comprises a plurality of grooves defined in its surface.

FIG. 8 is a magnified perspective view of an alternative embodiment of the liquid impermeable layer of the current invention, which comprises a plurality of vertical apertures defined through its surface.

FIG. 9 is a magnified cross section view of one embodiment of the liquid impermeable layer of the current invention, which comprises a plurality of channels defined in its surface.

FIG. 10 is a frontal perspective view of various shapes of the liquid impermeable layers of the current invention, where each shape comprises a different configuration of anti-microbial light strips.

FIG. 11 is a top-down plan view of the undergarment of the current invention wherein the relative location of a liquid impermeable layer is shown in broken line outline and where the undergarment comprises an inlet configured to deliver fluids to the liquid impermeable layer and an outlet configured to remove fluids from the liquid impermeable layer and into an external bag.

FIG. 12 is a frontal perspective of one embodiment of the liquid impermeable layer of the current invention where the liquid impermeable layer comprises an inlet configured to deliver fluids to a manifold in fluid contact with the liquid impermeable layer and an outlet configured to remove fluids from the manifold and into an external bag.

The disclosure and its various embodiments can now be better understood by turning to the following detailed description of the preferred embodiments, which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

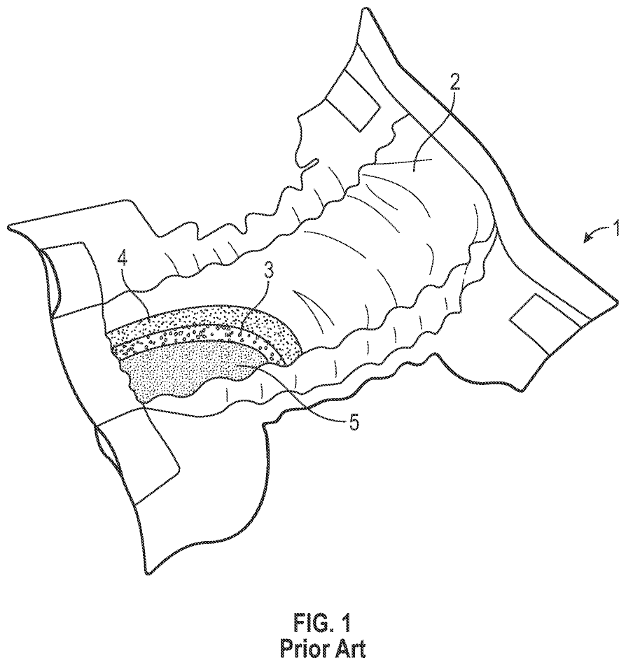

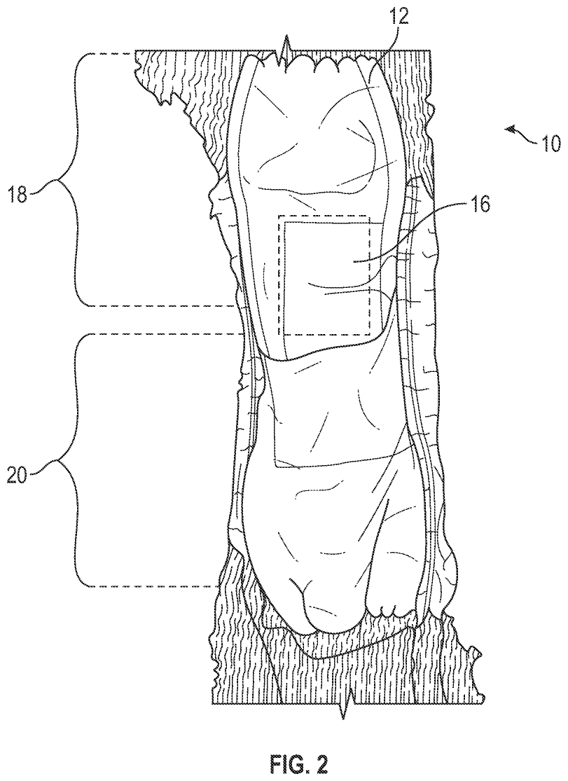

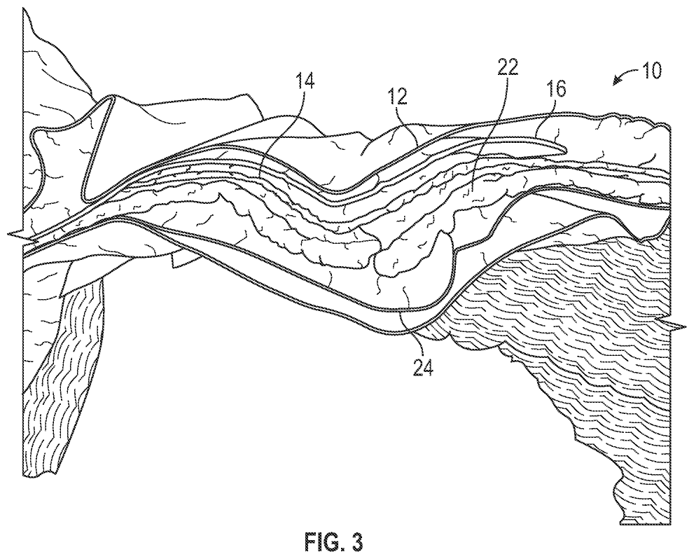

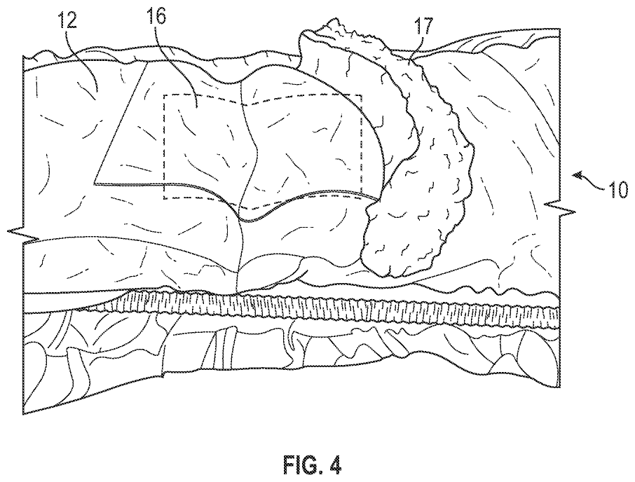

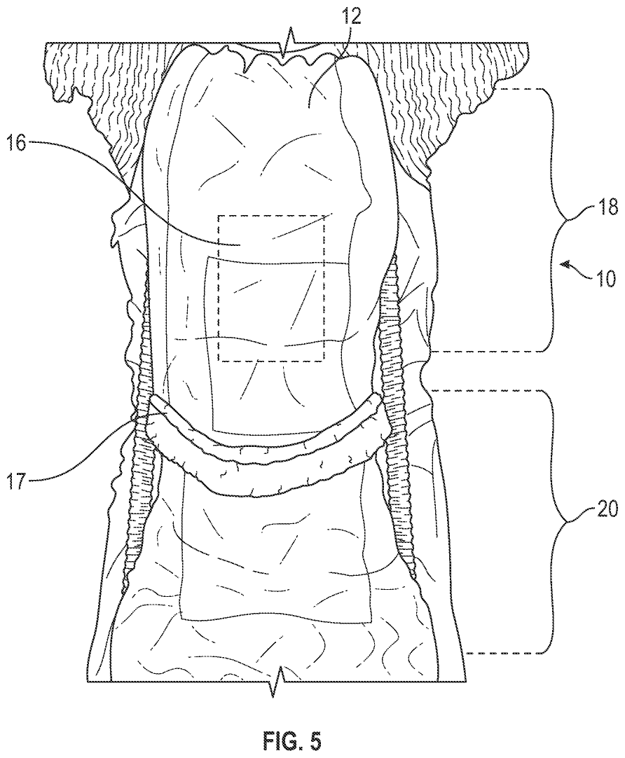

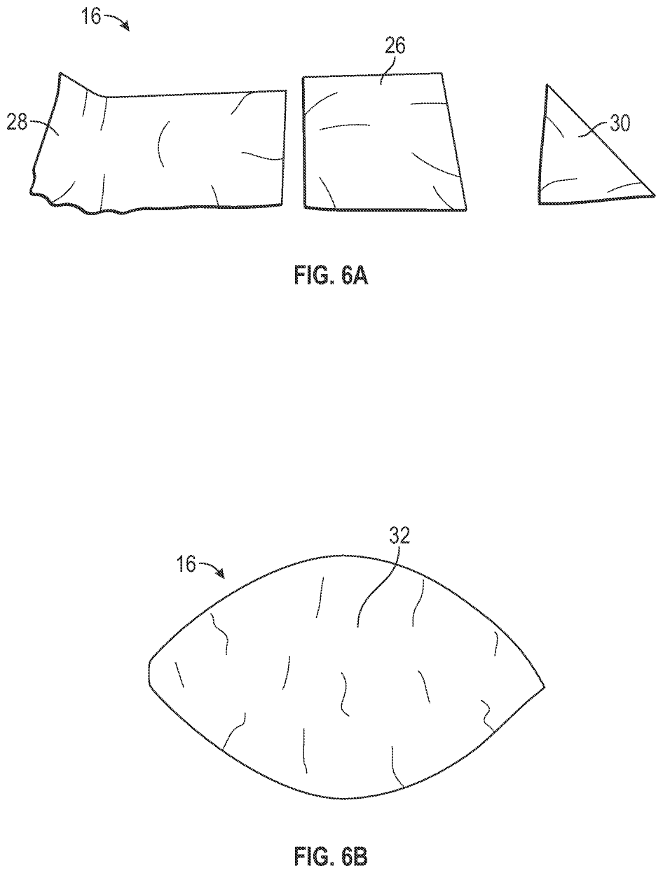

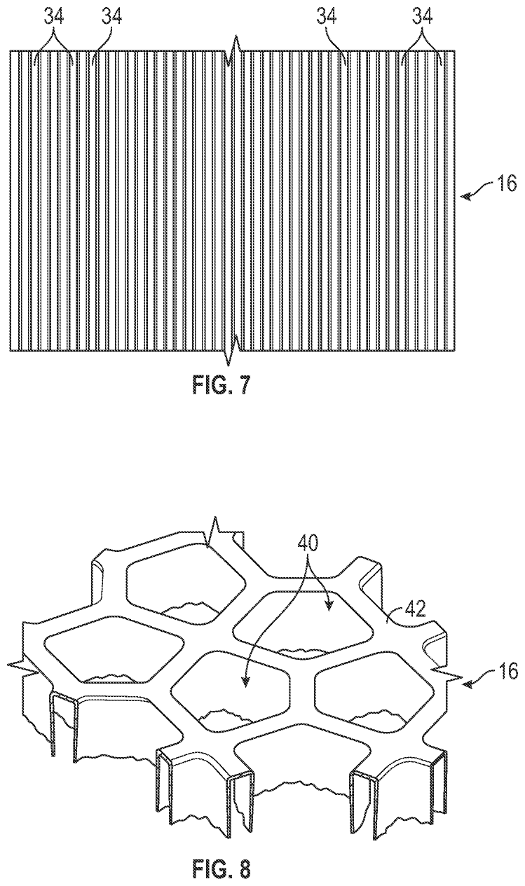

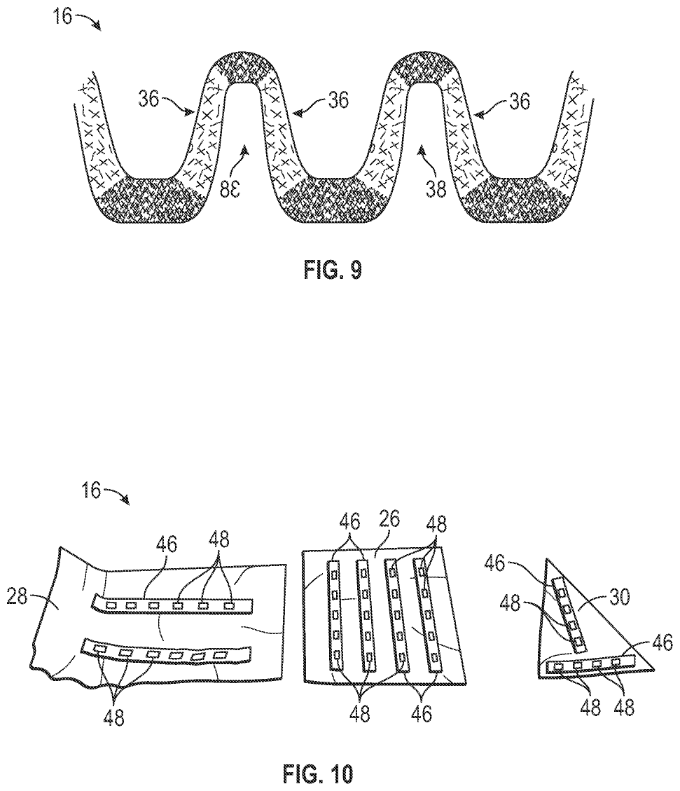

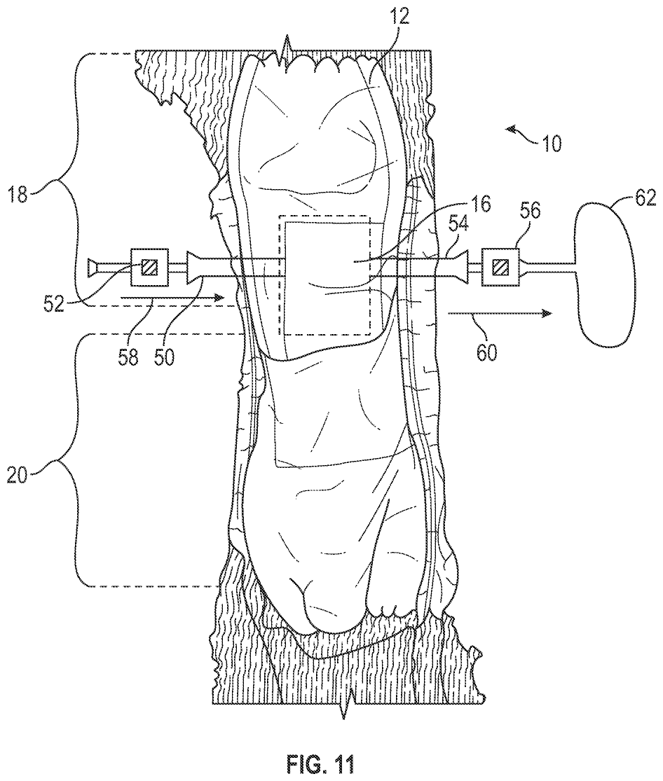

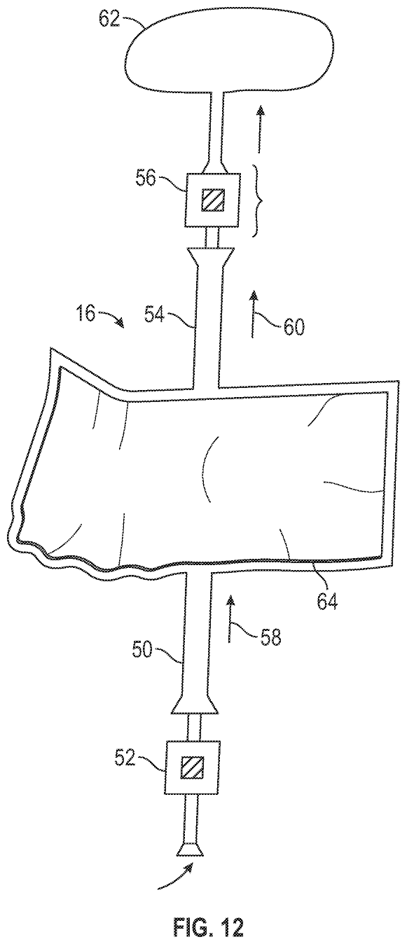

A standard or typical diaper as known in the art is seen in FIG. 1 and is denoted generally by reference numeral 1. In most instances, the diaper 1 comprises a topsheet 2, which is the upper most layer of the diaper 1 when in an open configuration as seen in FIG. 1. The topsheet 2 is the layer of the diaper 1 which makes contact with the wearer's skin and is specially designed to quickly transfer fluids to an absorbent core 3 while the topsheet 2 itself remains soft and dry to the touch. Immediately disposed under the topsheet 2 of the diaper 1 is an acquisition or distribution layer 4, which moves liquid away from the skin and distributes it more evenly across the entire surface area of the absorbent core 3 for better absorbency. The absorbent core 3 itself is the inner-most layer of the diaper 1 and typically consists of a blend of cellulose fluff pulp and sodium polyacrylate granules. The cellulose fluff pulp quickly absorbs and transfers urine to the polyacrylate superabsorbent material where it is trapped, thus helping to keep the skin dry, even if the wearer sits down or places pressure on a full diaper. The outermost layer of the diaper 1 comprises a backsheet 5 that is impermeable to all liquids and keeps the contents of the diaper 1 contained.

According to one embodiment, the current invention as seen in FIGS. 2 and 3, is an undergarment 10 comprising a topsheet 12 followed by an acquisition or distribution layer 14 with a liquid impermeable layer 16 disposed or inserted in between. Disposed beneath the distribution layer 14 is an absorbent core 22. A liquid impermeable bottom or backsheet 24 is in turn disposed on the opposing side of the absorbent core 22.

The undergarment 10 in FIG. 2 is seen with a portion of the topsheet 12 removed so as to illustrate the placement of the liquid impermeable layer 16 there beneath. The undergarment 10 in certain embodiments comprises the generally known form of a diaper, namely a wearable item comprising an inside surface or inner portion that makes contact with the wearer's skin, and an outside surface or outer portion that provides a barrier between the wearer and the outside environment. The undergarment 10 also comprises a front portion 18 and a rear portion 20, where the front portion 18 is configured to fit around or accommodate the pelvic region of the wearer and where the rear portion is configured to cover or accommodate the wearer's buttocks. The outside surface or outer portion typically also comprises a fastening means that attaches to another portion of the diaper so as to keep or maintain the diaper in a static position when being worn by the user. However, in certain other embodiments, the undergarment 10 may comprise other configurations, such as those associated with pads, sanitary napkins, or other wearable medical items. In certain embodiments, the liquid impermeable layer 16 is disposed immediately under the topsheet 12 and above the acquisition/distribution layer 14. In other embodiments, the liquid impermeable layer 16 is below or in line with the acquisition/distribution layer 14.

According to certain embodiments, the undergarment 10 comprises a stool blocker 17 as seen in FIGS. 4 and 5. The stool blocker 17 projects upward relative to the substantially horizontal surface of the topsheet 12 and is disposed substantially in the longitudinal center of the undergarment 10. In certain embodiments, the stool blocker 17 is disposed between or at the junction point between the front portion 18 and the back portion 20 of the undergarment 10. The stool blocker 17 is comprised of liquid or solid waste impermeable material and in some embodiments is comprised of the same material as the liquid impermeable layer 16. These materials include, but are not limited to, polyethylene, polyester, polypropylene, polyurethane, polyvinyl chloride, ethylene-vinyl acetate, polytetrafluoroethylene, expanded polytetrafluoroethylene, Gore-Tex®, or silicone, or any combination thereof. According to some embodiments, the stool blocker 17 is also comprised of any polymer, elastomer, rubber, composite, fabric, woven fiber, spunbound fiber, membrane, film, coating or layer that imparts a degree of liquid or waste impermeability or hydrophobicity, or both In other embodiments, the stool blocker 17 is not completely liquid or solid waste impermeable and is comprised of a mesh-like network of fibers that allow for some passage of waste into the mesh network and traps and stops the forward movement of solid waste. When the undergarment 10 is being worn, the stool blocker 17 is positioned between the wearer's urethra and anus so as to prevent stool or liquid collecting within the back portion 20 of the undergarment 10 from entering the front portion 18 of the undergarment 10. By maintaining stool or liquid within the back portion 20 of the undergarment 10, the stool blocker 17 assists the liquid impermeable layer 16 in keeping waste or other bacteria carrying media away from the wearer's urethra, thereby reducing the chances of infection.

The liquid impermeable layer 16 is impervious to liquids including but not limited to urine, water, stool, or any other liquid or semi-liquid substance and does not permit or allow the liquid to penetrate or access the portion of the distribution layer 14 that is disposed directly underneath the liquid impermeable layer 16 and vice versa. For example, according to certain embodiments, the liquid impermeable layer 16 is comprised of a material that does not allow for direct communication of excrement between the topsheet 12 of the undergarment 10 and the absorptive core 22 disposed below the liquid impermeable layer 16. In one particular embodiment, the material of the liquid impermeable layer 16 does not allow for the reabsorption of excrement held in the absorptive core 22 disposed below the liquid impermeable layer 16. In other embodiments the liquid impermeable layer 16 can have varying degrees of impermeability. The liquid impermeable layer in some embodiments can be gas permeable. In one particular embodiment, the liquid impermeable layer 16 is comprised of plastic or resin such as polyethylene; however in other embodiments, the liquid impermeable layer 16 is comprised of polyethylene, polyester, polypropylene, polyurethane, polyvinyl chloride, ethylene-vinyl acetate, polytetrafluoroethylene, expanded polytetrafluoroethylene, Gore-Tex®, or silicone, or any combination thereof. According to some embodiments, the liquid impermeable layer 16 is also comprised of any polymer, elastomer, rubber, composite, fabric, woven fiber, spunbound fiber, membrane, film, coating or layer that imparts a degree of liquid impermeability or hydrophobicity, or both. The layer 16 can be smooth, rough, textured, or patterned to aid in its function. The layer 16 can be elastic or rigid, compliant or non-compliant. According to certain embodiments, the liquid impermeable layer 16 is comprised of or covered with any other material now known or later devised which is liquid impermeable so that the urine or waste absorbed by the core 22 and any bacteria that is collected or present within the core 22 is unable to come into contact with the wearer's skin disposed directly above the liquid impermeable layer 16.

According to certain embodiments, the liquid impermeable layer 16 is configured to inherently repel or otherwise provide a hydrophobic surface. In one embodiment, the liquid impermeable layer 16 repels liquid based on the intrinsic properties of the material that comprises the liquid impermeable layer 16 itself; however in certain other embodiments, a coating, spray, or additional layer of material is applied to the liquid impermeable layer 16 which is configured to increase its hydrophobic properties. In certain other embodiments, the liquid impermeable layer 16 comprises an antibacterial, antiviral, bacteriostatic, bactericidal, antifungal, skin soothing or moisturizing, infection indicating, and/or other desirable substance incorporated into the material of the liquid impermeable layer 16 itself, or disposed as a coating or layer thereon.

According to certain embodiments, the liquid impermeable layer 16 is disposed in the front portion 18 of the undergarment 10. For example, in one particular embodiment seen in FIGS. 2, 4, and 5, the liquid impermeable layer 16 is disposed within the front portion 18 so as to be located below the female vulva, including the vaginal and urethral openings if the person wearing the undergarment 10 is female, and below the glans and urethra of the penis if the person wearing the undergarment 10 is male. Regardless of whether the person wearing the undergarment 10 is male or female, according to certain embodiments, the liquid impermeable layer 16 does not extend into or is disposed within the back portion 20 or any other portion of the undergarment 10 that receives stool. The liquid impermeable layer 16 is located below the wearer facing topsheet 12 and above the absorptive core 22; however, according to certain embodiments, the liquid impermeable layer 16 is above, below, in-line, or coplanar with the distribution layer 14.

According to certain embodiments, the shape of the liquid impermeable layer 16 is substantially square 26 shaped; however, in certain other embodiments the liquid impermeable layer 16 comprises a substantially oblong 32, rectangular 28, or triangular 30 shape as seen in FIGS. 6A and 6B. In one particular embodiment, the liquid impermeable layer 16 comprises any shape so long as it covers the entire length and width of the female vulva, including the vaginal and urethral opening, and the glans and urethra of the male penis as discussed above.

In certain embodiments, the liquid impermeable layer 16 comprises a size that is large enough so as to extend or overlap beyond the vaginal and urethral opening and the glans and urethra of the male penis of the wearer. The liquid impermeable layer 16 extends beyond the anatomical features of the wearer so as to prevent excrement and the bacteria that may be contained therein that has been absorbed by the core 22 from making contact with the wearer. For example, the liquid impermeable layer 16 comprises a longitudinal length that is up to three inches long or which is otherwise sufficiently long enough to cover or accommodate the vaginal and/or urethral openings of the wearer. In certain embodiments the liquid impermeable layer 16 comprises a lateral width that is up to two inches wide. According to some embodiments, the dimensions of the liquid impermeable layer 16 will vary depending on the wearers age and garment size, with iterations for children and infants being substantially smaller to accommodate the anatomy of the wearer. The dimensions will also vary depending on the size of diaper, and in some embodiments the liquid impermeable layer 16 comprises a longitudinal length that is larger or smaller than the example provided of three inches long and a lateral width that is larger or smaller than the example provided of up to two inches wide.

In certain embodiments, the liquid impermeable layer 16 does not span the entire width or length of the undergarment 10, thereby providing or maintaining a border area or portion adjacent to the liquid impermeable layer 16 for direct fluidic communication between either the topsheet 12 or the acquisition/distribution layer 14 and the absorbent core 22 disposed beneath. In an alternative embodiment, the liquid impermeable layer 16 spans across an entire lateral width of the undergarment 10 so that urine and excrement making contact with the liquid impermeable layer 16 flows or drains off either the frontal or rear edges of the liquid impermeable layer 16. In an alternative embodiment, the liquid impermeable layer 16 spans across an entire length from the midline of the undergarment 10 to the front so that urine and excrement making contact with the liquid impermeable layer 16 flows or drains off either the lateral edges or the posterior edge of the liquid impermeable layer 16. In no instance however does the liquid impermeable layer 16 comprise a longitudinal length that extends into a region of the back portion 20 which receives stool from the wearer.

According to certain embodiments, the upward facing surface of the liquid impermeable layer 16 comprises a plurality of groves, ridges, pores, or channels to aid in removing urine from the wearer and directing it to the absorption core 22 disposed below. In other instances, the upward facing surface of the liquid impermeable layer 16 is flat with no groves, ridges, pores, or channels. FIG. 7 is a magnified view of one exemplary embodiment in which the liquid impermeable layer 16 comprises a plurality of vertically orientated grooves 34 defined across an upward facing surface. FIG. 9 is a magnified cross-sectional view of one embodiment of the liquid impermeable layer 16 where layer 16 is comprised of a plurality of upward facing channels 36 separated by a corresponding plurality of opposing downward facing troughs 38, where the upward facing channels 36 are configured to direct urine and waste therein, and where the downward facing troughs 38 provide additional negative space or volume between the liquid impermeable layer 16 and the absorbent core 22 disposed beneath. In one particular embodiment, each of the plurality of contours 34 and 36 are comprised of the same material as the liquid impermeable layer 16 outlined above and do not absorb any excrement received from the wearer According to one embodiment, the plurality of groves 34 or channels 36 are also disposed on the bottom surface of the liquid impermeable layer 16 facing the absorbent core 22. In certain other embodiments, the plurality of contours run horizontally or diagonally along a horizontal axis of the liquid impermeable layer 16 and assist in the transportation or direction of urine and excrement into the absorptive core 22 below. According to certain other embodiments, the liquid impermeable layer 16 comprises additional means for directing urine or excrement, including, but not limited to, capillary action, directional pores, one-way valves, hydrophobic coatings, chemical coatings, or other known methods.

According to certain embodiments, the liquid impermeable layer 16 comprises a plurality of vertically configured or stacked funnels, grooves, baffles, capillaries, slots, holes, fissures, channels, pores, one-way valves, or other contoured pattern that is configured to direct or drain the excrement away from the skin of the wearer. Each of the plurality of vertical funnels comprises the same material as the liquid impermeable layer 16 itself so that the excrement is not absorbed as the excrement passes through. For example, as seen in FIG. 8, at least one portion or layer of the liquid impermeable layer 16 comprises a plurality of apertures or passages 40 defined within a honeycomb-like structure 42, each of the plurality of apertures or passages 40 being configured to direct urine and waste through to another portion of the liquid impermeable layer 16 below. In a related embodiment, the liquid impermeable layer 16 comprises a plurality of layers, where each of the plurality of layers comprises at least one vertically connected contour or channel. The bottommost layer is impermeable to all liquid and reabsorption from below and will therefore allow the collected urine and excrement to roll or move off of the lateral edges of the liquid impermeable layer 16 and into the liquid absorptive core 22 below. According to certain embodiments, the liquid impermeable layer 16 comprises both horizontal and vertical contours.

In yet another embodiment, the liquid impermeable layer 16 comprises means for visible light phototherapy to act as an antibacterial and antimicrobial agent. As seen in FIG. 10, the visible light phototherapy is delivered using at least one thin strip 46, the strip 46 itself comprising a plurality of light emitting diodes (LEDs) 48. Each strip 46 is flexible and waterproof and is disposed on, embedded into, or otherwise coupled to the bottom surface of the impermeable layer 16. Each LED 48 is configured to provide light in various wavelengths that kill microorganisms. The light intermittently or constantly illuminates the impermeable layer 16, topsheet 12, and/or the absorbent core 22 to reduce microbial growth thereon. According to one embodiment, each strip 46 is disposable or reusable and comprises a small battery component. According to one embodiment, each strip 46 is disposed on the bottom of the liquid impermeable layer 16 and is oriented or directed downward towards the absorbent core 22. In an alternative embodiment, the impermeable layer 16 comprises a plurality of strips 46, where each strip 46 comprises a separate plurality of anti-microbial LEDs 48 that are directed both upwards towards the topsheet 12, and simultaneously downwards towards to absorbent core 22 below to provide anti-microbial light in both directions. Each strip 46 is oriented so as to either entirely cover or partially cover the bottom of the liquid impermeable layer 16.

According to certain embodiments, the liquid impermeable layer 16 comprises a plurality of strips 46 disposed in parallel with each other, for example as seen in FIG. 10 where the rectangular 28 embodiment comprises a pair of strips 46 disposed in parallel relative to each other, and where the square 26 embodiment comprises at least four strips 46, each strip 46 being parallel to each other. In a related embodiment, the liquid impermeable layer 16 comprises a plurality of strips 46 disposed in a different configuration, for example where each strip 46 is perpendicular to each other as seen on the triangular 30 embodiment in FIG. 10. It is to be expressly understood that the number or positioning of the plurality of strips 46 as seen in FIG. 10 is meant to be illustrative purposes only and that fewer or additional strips 46 may be used in alternative configurations without departing from the original spirit and scope of the current invention.

In a further embodiment seen FIGS. 11 and 12, the liquid impermeable layer 16 comprises an inlet 50 and a first connector 52 configured to deliver at least one anti-microbial substance or drying material to the liquid impermeable layer 16 in order to reduce bacterial growth and promote dryness. An outlet 54 and a second connector 56 are disposed on an opposing lateral side of the liquid impermeable layer 16 relative to the inlet 50 and the first connector 52, the outlet 54 and second connector 56 being configured to remove any discarded anti-microbial substances. In some embodiments, the antimicrobial substance that is delivered to the liquid impermeable layer 16 comprises antimicrobial cleansers, disinfectant, ozone, or air to keep the liquid impermeable layer 16 dry. According to one embodiment, the first and second connectors are each a leur connector, or other connector, disposed on the exterior of the undergarment 10 and coupled to the liquid impermeable layer 16 via the inlet 50 and outlet 54, respectively. The first connector allows for the selective passage of liquid or gas material through the inlet 50 in the direction denoted by arrow 58 to make contact with the liquid impermeable layer 16 with the purpose of drying or sanitizing the liquid impermeable layer 16. Once used, the fluid or gas passes through the outlet 54 in the direction denoted by arrow 60, through the second connector 56, and exits into an exterior bag 62 and away from the wearer. In certain embodiments, the second connector comprises an external pH sensor or bacterial sensor so as to quickly assess for bacteria or infection. According to certain embodiments, both the inlet 50 and the outlet 54 are each comprised of a plurality of sub-tubes or sub-channels that are each in fluid communication with the liquid impermeable layer 16.

In a related embodiment seen in FIG. 12, after the anti-microbial substance passes through the first connector 52 and the inlet 50, it enters a manifold 64 that is in fluid contact with at least one surface of the liquid impermeable layer 16. The manifold 64 accommodates at least one surface of the liquid impermeable layer 16 so that the entire surface comes into contact with anti-microbial or drying substance. After passing over the liquid impermeable layer 16, the now used anti-microbial/drying substance exits the manifold 64 and enters the external bag 62 after passing through the outlet 54 and second connector 56.

The liquid impermeable layer 16 prevents urine, fluid, and any other media capable of transporting and breeding bacteria that is deposited into the undergarment 10 from contacting the skin of the person wearing the undergarment 10. According to certain embodiments, fluid that is released from the wearer makes contact with the topsheet 12 where it then quickly transfers to the upward or top facing surface of the liquid impermeable layer 16. The curvature or shape of the liquid impermeable layer 16 assists in directing the fluid away from the skin of the wearer and towards the outer edges of the liquid impermeable layer 16, which comprises a smaller surface area than the topsheet 12. In certain embodiments, the liquid impermeable layer 16 comprises a plurality of grooves 34 and/or channels 36, which assist in directing the fluid towards to outer edges of the liquid impermeable layer 16. In certain other embodiments, the liquid impermeable layer 16 itself comprises a plurality of layers so as to further draw fluid away from wearer's skin, for example, through a plurality of passages 40 defined therein, before at least one of the plurality layers then directs the fluid towards the edges of the liquid impermeable layer 16. Once reaching an edge of the liquid impermeable layer 16, the fluid is then absorbed and held within the absorbent core 22. According to certain embodiments, the distribution layer 14 further spreads or directs the fluid away from the liquid impermeable layer 16 so that it may be absorbed by the core 22. Over time, as the core 22 absorbs more and more fluid, the liquid impermeable layer 16 prevents the fluid from touching the wearer's skin and anatomy directly above the layer 16, even when the core 22 has become saturated. By maintaining the fluid within the absorbent core 22, the liquid impermeable layer 16 prevents the fluid and any bacteria therein from making sustained contact with the wearer's anatomy and thus drastically lowers the probability of infection or other complications. When saturated, the previous undergarment 10 is removed from the wearer and a fresh, new undergarment 10 is applied, allowing the process to continue.

It has long been known that urine and human waste in general is a breeding ground for bacteria and other pathogens, which, for example, can cause urinary tract infections, bladder infections, skin related rashes, and other undesirable conditions. It therefore follows that by reducing the amount of exposure a wearer has to such bacteria, the lower the probability that such infections or complications will occur. The undergarment 10 of the current invention accomplishes this by using the liquid impermeable layer 16 to direct urine and other waste away from the wearer's skin and into the absorbent core 22 disposed beneath and prevent any backflow of moisture, waste, and bacteria from the core from reaching the wearer's skin. However, what has been surprising is the degree to which the current undergarment 10 reduces the amount of harmful bacteria exposure to the wearer.

For example, a first experiment was performed to examine the reduction of growth of bacteria on the portion of the undergarment 10 comprising the liquid impermeable layer 16 that is disposed under the vaginal and urethral opening and the glans and urethra of the male penis of the wearer, versus the reduction of growth of bacteria on a portion of the undergarment 10 with no liquid impermeable layer 16. The garment used for testing comprised a topsheet, followed by a liquid impermeable layer 16 disposed in the front portion 18 of the diaper, followed by a distribution layer 14, and the absorbent core 22. Test cultures to simulate urine or liquid waste were initiated in Tryptic Soy Broth (TSB) and allowed to incubate under conditions necessary for sufficient growth prior to testing. The inoculum was prepared from the test culture by pelleting and resuspending the individual cultures in phosphate buffered saline (PBS). The suspensions were then pooled together into a single conical. The conical was supplemented with PBS to reach a total inoculum volume of approximately 50 ml.

The undergarment 10 comprising the liquid impermeable layer 16 was placed onto a clean tray that was large enough to hold the article. The area of the undergarment 10 with the liquid impermeable layer 16 and the area of the undergarment 10 with no liquid impermeable layer were each inoculated with 15 mL of inoculum. This inoculum was a liquid medium simulating urine or liquid waste with three UTI causing bacteria, namely Escherichia coli, Klebsiella pneumoniae, and Pseudomonas aeruginosa. A layer of five individual sterile polyester swatches were placed into a single petri dish to serve as the control. The polyester swatches were inoculated with 15 ml of inoculum. The petri dish was closed and sealed with parafilm. The undergarment 10 and control were then placed into a humid environment and incubated at the appropriate temperature for eight hours. After incubation, the control was aseptically transferred to a neutralizer tube, serially diluted, then plated onto Tryptic Soy Agar (TSA). The inoculated areas of the undergarment 10 were aseptically cut into approximately 2″×2″ swatches. The top two layers of the swatches were aseptically transferred into individual neutralizer tubes, serially diluted, and then plated onto TSA. The plates were incubated at the appropriate temperature for the duration of time necessary for proper growth. Following incubation, the agar plates were counted, and the results were recorded as CFU/Carrier. The results provided in Table 1 below found that compared to the diaper without the liquid impermeable layer 16 of the current invention, the portions of the wearer facing undergarment 10 disposed above the liquid impermeable layer 16 had a 99.72% reduction in growth of the bacteria, an amount that was previously unexpected.

TABLE 1

| | | | | | |
|---|---|---|---|---|---|
| [symbol] | [symbol] | [symbol] | [symbol] | [symbol] | [symbol] |

| | | | | |
|---|---|---|---|---|
| [symbol] | Polyester | [symbol] | [symbol] | N/A |

| | | | | |
|---|---|---|---|---|
| Polyester | [symbol] | [symbol] | | |
| [symbol] | [symbol] | [symbol] | | |
| [symbol] | Test | [symbol] | 99.72% | 2.56 |

[symbol]

[symbol]

indicates data missing or illegible when filed

A second experiment was performed in order to examine the growth of the same bacteria over a portion of the undergarment 10 comprising the liquid impermeable layer 16 and an area of the undergarment 10 with no liquid impermeable layer using a gel medium to simulate stool.

Test cultures were initiated in TSB and allowed to incubate under conditions necessary for sufficient growth prior to testing. The inoculum was prepared from the test culture by pelleting and resuspending the individual cultures in PBS. The suspensions were then pooled together into a single conical. The conical was supplemented with 50% TSA and vortexed to homogeneously mix the pooled inoculum and 50% TSA to simulate a stool sample. The undergarment 10 was placed onto a clean tray that was large enough to hold the article. The area of the undergarment 10 with the liquid impermeable layer 16 and the area of the undergarment 10 with no liquid impermeable layer were each inoculated with 15 mL of simulated stool sample. A layer of five individual sterile polyester swatches were placed into a single petri dish to serve as the provided control. The polyester swatches were inoculated with 15 ml of simulated stool sample. The petri dish was closed and sealed with parafilm. The test undergarment 10, control diaper and control were then placed into a humid environment and incubated at the appropriate temperature for eight hours on an oscillator to simulate movement. After incubation, the control was aseptically transferred to a neutralizer tube, serially diluted, then plated onto TSA. The inoculated areas of the undergarment 10 were scraped to remove the simulated stool inoculum and were then aseptically cut into approximately 2″×2″ swatches. The top two layers of the swatches were aseptically transferred into individual neutralizer tubes, serially diluted, then plated onto TSA. The plates were incubated at the appropriate temperature for the duration of time necessary for proper growth. Following incubation, the agar plates were counted, and the results were recorded as CFU/Carrier. The results provided in Table 2 below found that compared to the diaper without the liquid impermeable layer 16 of the current invention, the portions of the wearer facing undergarment 10 that were disposed above the liquid impermeable layer 16 had a 50.43% reduction in growth of the bacteria, an amount that was previously unexpected and further indication that, even in instances where stool crosses over the stool blocker 17 or otherwise makes contact with the liquid impermeable layer 16, the person wearing the undergarment 10 of the current invention still incurs a substantial benefit in reducing the likelihood of bacterial infection.

TABLE 2

| | | | | | |
|---|---|---|---|---|---|
| [symbol] | [symbol] | [symbol] | [symbol] | [symbol] | [symbol] |

| | | | | |
|---|---|---|---|---|
| [symbol] | Polyester | [symbol] | [symbol] | N/A |

| | | | | |
|---|---|---|---|---|
| Polyester | [symbol] | [symbol] | | |
| [symbol] | [symbol] | [symbol] | | |
| [symbol] | Test | [symbol] | 50.43% | 0.30 |

[symbol]

indicates data missing or illegible when filed

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments include other combinations of fewer, more, or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material, or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structures, material, or acts for performing substantially the same function in substantially the same way to obtain substantially the same result. Therefore, it is contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can, in some cases, be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

1. A removable undergarment for directing fluid away from a user, the undergarment comprising:
a topsheet;
an absorbent core; and
a liquid impermeable layer disposed beneath the topsheet and above the absorbent core,
wherein the liquid impermeable layer is only disposed beneath a portion of the topsheet and above a portion of the absorbent core which corresponds to a urethra of the user when the undergarment is worn by the user, and
wherein the liquid impermeable layer is configured to prevent bacteria disposed within the portion of the absorbent core which corresponds to the urethra of the user when the undergarment is worn by the user from contacting the urethra of the user.

2. The undergarment of claim 1, where the liquid impermeable layer comprises a plurality of grooves or channels configured to direct fluid to at least one outer edge of the liquid impermeable layer.

3. The undergarment of claim 1, where the liquid impermeable layer is at least partially impermeable to liquid.

4. The undergarment of claim 1, where the liquid impermeable layer is at least partially hydrophobic.

5. The undergarment of claim 1 where the liquid impermeable layer comprises of at least one of the following: polyethylene, polyester, polypropylene, polyurethane, polyvinyl chloride, ethylene-vinyl acetate, polytetrafluoroethylene, expanded polytetrafluoroethylene, Gore-Tex®, silicone, or any polymer, elastomer, rubber, composite, fabric, woven fiber, spunbound fiber, membrane, film, coating or a layer comprising a degree of liquid impermeability or hydrophobicity, or both.

6. The undergarment of claim 1, where the liquid impermeable layer comprises at least one of the following: antibacterial material, antiviral material, bacteriostatic material, bactericidal material, antifungal material, moisturizing material, or infection indicating material.

7. The undergarment of claim 1, where the liquid impermeable layer comprises at least one light source configured to provide microbe killing visible light, wherein the light intermittently or constantly illuminates the topsheet, the impermeable layer, or the absorbent core.

8. The undergarment of claim 1, where the liquid impermeable layer comprises an inlet and a first connector configured to deliver an anti-microbial substance or drying material to the liquid impermeable layer and an outlet and a second connector configured to transport the anti-microbial substance or drying material out the undergarment and into an external bag.

9. The undergarment of claim 1, where the liquid impermeable layer is disposed within a front portion of the undergarment.

10. The undergarment of claim 9 further comprising a stool blocker disposed between the front portion of the undergarment and a back portion of the undergarment.

11. The undergarment of claim 1, where the liquid impermeable layer comprises a shape configured to only be disposed directly beneath the vaginal and urethral openings if the person wearing the undergarment is female and directly beneath the glans and urethra of the penis if the person wearing the undergarment is male.

12. The undergarment of claim 1 further comprising a border area adjacent to the liquid impermeable layer, the border area providing direct fluid communication between the topsheet and the absorbent core.

13. The undergarment of claim 1, where the liquid impermeable layer comprises at least one aperture defined therein, with at least one aperture configured to direct fluid through the liquid impermeable layer.

14. The undergarment of claim 1 further comprising a distribution layer disposed between the liquid impermeable layer and the absorbent core.

15. The undergarment of claim 1 further comprising a distribution layer disposed between the topsheet and the liquid impermeable layer.

16. The undergarment of claim 1 further comprising a distribution layer disposed coplanar with the liquid impermeable layer.

17. A method for reducing bacterial infections in a person wearing a removable undergarment, the method comprising:
contacting a topsheet of the undergarment to a portion of the wearer's anatomy;
absorbing a fluid received from the wearer through the topsheet;
directing the received fluid across a surface of a liquid impermeable layer and to at least one outer edge of the liquid impermeable layer; and
absorbing the fluid within a core disposed beneath the liquid impermeable layer.

18. The method of claim 17 where directing the received fluid across the surface of the liquid impermeable layer comprises directing the received fluid through at least one grove or channel defined on the surface of the liquid impermeable layer.

19. The method of claim 17 where directing the received fluid across the surface of the liquid impermeable layer comprises directing the received fluid across a front portion of the undergarment.

20. The method of claim 17 where directing the received fluid to at least one outer edge of the liquid impermeable layer comprises directing the received fluid to a border area adjacent to the liquid impermeable layer and beneath the topsheet.

21. The method of claim 17 where directing the received fluid across the surface of the liquid impermeable layer comprises directing the received fluid through at least one aperture defined through the liquid impermeable layer.

22. The method of claim 17 further comprising blocking stool received from the wearer from entering a front portion of the undergarment.

23. The method of claim 17 further comprising preventing fluid absorbed by the core from recontacting the topsheet in contact with the portion of the wearer's anatomy.

24. The method of claim 17 further comprising distributing the fluid over a surface of the core after it has been directed off of the at least one outer edge of the liquid impermeable layer.

25. The method of claim 17 further comprising distributing the received fluid over the surface of the liquid impermeable layer after being absorbed through the topsheet.
